(19) United States
(12) Patent Application Publication
(10) Pub. No.: US 2008/0021522 A1
(43) Pub. Date: Jan. 24, 2008

(54) TELEMETRY ANTENNAS FOR IMPLANTABLE MEDICAL DEVICES

(75) Inventors: William D. VERHOEF, Andover, MN (US); Piotr PRZYBYSZEWSKI, Coon Rapids, MN (US); Christopher C. FULLER, Bloomington, MN (US); Gregory J. HAUBRICH, Champlin, MN (US); Garry L. DUBLIN, Maple Grove, MN (US); David NGHIEM, Shoreview, MN (US); Rodney S. WALLACE JR., Maple Grove, MN (US)

Correspondence Address: INGRASSIA FISHER & LORENZ, P.C., 7150 E. CAMELBACK, STE. 325, SCOTTSDALE, AZ, 85251, US

(73) Assignee: MEDTRONIC, INC., MINNEAPOLIS, MN (US)

(21) Appl. No.: 11/772,994

(22) Filed: Jul. 3, 2007

Related U.S. Application Data
(63) Continuation of application No. 11/116,691, filed on Apr. 28, 2005, now abandoned.

(51) Int. Cl. A61N 1/00

(52) U.S. Cl. 607/60

(57) ABSTRACT

An implantable medical device (“IMD”) configured in accordance with an example embodiment of the invention generally includes a housing, a connector header block coupled to the housing, and a telemetry antenna located within the header block. The header block is formed from a dielectric material, which encapsulates the antenna. The antenna is configured to support far field telemetry with an external device such as a programmer. In one example embodiment, the antenna is formed from a thin round wire, has a feed point on the top perimeter sidewall of the housing, and has a floating endpoint in the header block. The antenna is contoured to form a simple curve in a plane that is approximately parallel with the major sides of the housing.

### CROSS-REFERENCE TO RELATED APPLICATION

This application is a continuation of U.S. patent application Ser. No. 11/116,691, filed Apr. 28, 2005.

### TECHNICAL FIELD

The present invention relates generally to implantable medical devices (“IMDs”). More particularly, the present invention relates to telemetry antennas suitable for deployment in IMDs.

### BACKGROUND

It is well known in the art that IMDs provide diagnostic and/or therapeutic capabilities. Such IMDs include, without limitation: cardiac pacemakers; implantable cardioverters/defibrillators (“ICDs”); and various tissue, organ, and nerve stimulators or sensors. IMDs typically include functional components contained within a hermetically sealed enclosure or housing, which is sometimes referred to as a “can.” In some IMDs, a connector header or connector block is attached to the housing, and the connector block facilitates interconnection with one or more elongated electrical medical leads.

The header block is typically molded from a relatively hard, dielectric, non-conductive polymer having a thickness approximating the thickness of the housing. The header block includes a mounting surface that conforms to, and is mechanically affixed against, a mating sidewall surface of the housing.

It has become common to provide a communication link between the hermetically sealed electronic circuitry of the IMD and an external programmer, monitor, or other external medical device (“EMD”) in order to provide for downlink telemetry transmission of commands from the EMD to the IMD and to allow for uplink telemetry transmission of stored information and/or sensed physiological parameters from the IMD to the EMD. As the technology has advanced, IMDs have become more complex in possible programmable operating modes, menus of available operating parameters, and capabilities of monitoring, which in turn increase the variety of possible physiologic conditions and electrical signals handled by the IMD. Consequently, such increasing complexity places increasing demands on the programming system.

Conventionally, the communication link between the IMD and the EMD is realized by encoded radio frequency (“RF”) transmissions between an IMD telemetry antenna and transceiver and an EMD telemetry antenna and transceiver. The telemetry transmission system that evolved into current common use relies upon the generation of low amplitude magnetic fields by current oscillating in an LC circuit of an RF telemetry antenna in a transmitting mode and the sensing of currents induced by a closely spaced RF telemetry antenna in a receiving mode. Short duration bursts of the carrier frequency are transmitted in a variety of telemetry transmission formats. In some products, the RF carrier frequency is set at 175 kHz, and the prior art contains various RF telemetry antenna designs suitable for use in such applications. To support such products, the EMD is typically a programmer having a manually positioned programming head having an external RF telemetry antenna. Generally, the IMD antenna is disposed within the hermetically sealed housing, however, the typically conductive housing adversely attenuates the radiated RF field and limits the data transfer distance between the programmer head and the IMD RF telemetry antennas to a few inches.

The above-described telemetry system employing the 175 kHz carrier frequency limits the upper data transfer rate, depending upon bandwidth and the prevailing signal-to-noise ratio. Using prior art RF telemetry antennas may result in: (1) a very low radiation efficiency due to feed impedance mismatching and ohmic losses; (2) a radiation intensity that is attenuated in an undesirable manner; and/or (3) poor noise immunity due to the distance between, and poor coupling of, the receiver and transmitter RF telemetry antenna fields.

It has been recognized that “far field” telemetry, or telemetry over distances of a few to many meters from an IMD, would be desirable. Various attempts have been made to provide antennas with an IMD to facilitate far field telemetry. Many proposals have been advanced for eliminating conventional RF telemetry antenna designs and substituting alternative telemetry transmission systems and schemes employing far higher carrier frequencies and more complex signal coding to enhance the reliability and safety of the telemetry transmissions while increasing the data rate and allowing telemetry transmission to take place over a matter of meters rather than inches. A number of alternative IMD telemetry antennas mounted outside of the hermetically sealed housing have been proposed. These approaches may be undesirable in that, depending upon the option selected, they may require substantial modification of the housing and/or header block, require additional components added to the housing, reduce the effectiveness of other components (e.g., reducing the available surface area of the can for use as a ground plane or electrode), create a directional requirement (e.g., require that the IMD be oriented in a particular direction during implant for telemetry effectiveness), or add extraneous exposed components that are subject to harmful interaction in the biological environment or require additional considerations during implant (e.g., stub antennas extending outward from the device).

It remains desirable to provide a far field telemetry antenna for an IMD that eliminates drawbacks associated with the IMD telemetry antennas of the prior art. Furthermore, other desirable features and characteristics of the present invention will become apparent from the subsequent detailed description and the appended claims, taken in conjunction with the accompanying drawings and the foregoing technical field and background.

### BRIEF SUMMARY

An IMD configured in accordance with an embodiment of the invention includes a far field telemetry antenna that is encapsulated within the header block of the IMD. The antenna topology is selected to fit within the physical space limitations of the header block while providing acceptable RF performance characteristics. In a practical embodiment of the invention, the antenna is conformal such that it has a minimal impact on the IMD volume. The antenna may be optimized to suit the needs of the particular IMD application, e.g., in consideration of the operating environment, the age, sex, or condition of the patient, or implant orientation within the patient.

The above and other aspects of the invention may be carried out in one form by an IMD comprising a housing, a header block coupled to the housing, and a simple-curved antenna located within the header block, where the antenna has a feed point from the housing leading into the header block, and a floating endpoint in the header block.

### BRIEF DESCRIPTION OF THE DRAWINGS

A more complete understanding of the present invention may be derived by referring to the detailed description and claims when considered in conjunction with the following figures, wherein like reference numbers refer to similar elements throughout the figures.

FIG. 1 is a perspective view of an IMD;

FIG. 2 is a schematic representation of an IMD and functional elements associated with the IMD;

FIG. 3 is a side view of an IMD configured in accordance with one embodiment of the invention;

FIG. 4 is a top view of the header block portion of the IMD shown in FIG. 3;

FIG. 5 is a cross sectional view of the header block portion of the IMD shown in FIG. 3, as viewed along line A-A in FIG. 4;

FIG. 6 is a cross sectional view of a header block portion of an IMD configured in accordance with an alternate embodiment of the invention;

FIG. 7 is a cross sectional view of a header block portion of an IMD configured in accordance with another alternate embodiment of the invention;

FIG. 8 is a side view of an IMD configured in accordance with another alternate embodiment of the invention;

FIG. 9 is a top view of the header block portion of the IMD shown in FIG. 8;

FIGS. 10-15 are top views of the header block portion of IMDs configured in accordance with alternate embodiments of the invention;

FIG. 16 is a side view of an IMD configured in accordance with an alternate embodiment of the invention;

FIG. 17 is a top view of the header block portion of the IMD shown in FIG. 16;

FIG. 18 is a cross sectional view of the header block portion of the IMD shown in FIG. 16, as viewed along line B-B in FIG. 17;

FIG. 19 is a side view of an IMD configured in accordance with another alternate embodiment of the invention;

FIG. 20 is a top view of the header block portion of the IMD shown in FIG. 19; and

FIG. 21 is a cross sectional view of the header block portion of the IMD shown in FIG. 19, as viewed along line C-C in FIG. 20.

### DETAILED DESCRIPTION

The following detailed description is merely illustrative in nature and is not intended to limit the invention or the application and uses of the invention. Furthermore, there is no intention to be bound by any expressed or implied theory presented in the preceding technical field, background, brief summary or the following detailed description.

The following description refers to components or features being “connected” or “coupled” together. As used herein, unless expressly stated otherwise, “connected” means that one component/feature is directly or indirectly connected to another component/feature, and not necessarily mechanically. Likewise, unless expressly stated otherwise, “coupled” means that one component/feature is directly or indirectly coupled to another component/feature, and not necessarily mechanically. Thus, although the figures may depict example arrangements of elements, additional intervening elements, devices, features, or components may be present in an actual embodiment (assuming that the functionality of the IMDs are not adversely affected).

The invention relates to an improved RF telemetry antenna for an IMD. The following description addresses various embodiments in the context of an ICD. However, the invention is intended to be implemented in connection with a wide variety of IMDs. For the sake of brevity, conventional techniques related to RF antenna design, IMD telemetry, RF data transmission, signaling, IMD operation, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in a practical embodiment.

An IMD antenna has two primary functions: to convert the electromagnetic power of a downlink telemetry transmission of an EMD telemetry antenna propagated through the atmosphere (and then through body tissues) into a UHF signal that can be processed by the IMD transceiver into commands and data that are intelligible to the IMD electronic operating system; and to convert the uplink telemetry UHF signals of the IMD transceiver electronics into electromagnetic power propagated through the body tissue and the atmosphere so that the EMD can receive the signals.

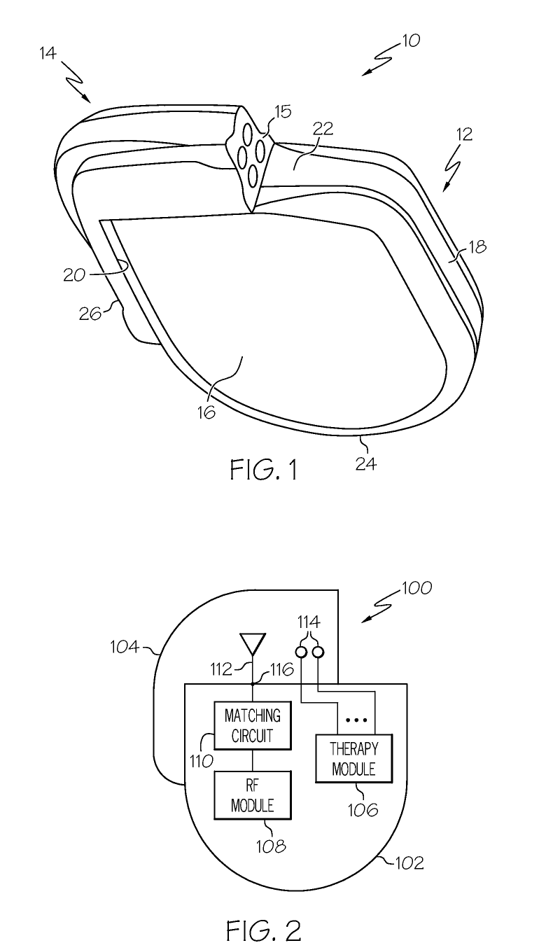

FIG. 1 is a perspective view of an IMD 10 having a hermetically sealed housing 12 and a connector header or block 14. A set of IMD leads having electrodes (such as cardioversion/defibrillation electrodes and pace/sense electrodes) disposed in operative relation to a patient's heart are adapted to be coupled to the header block 14 in a manner well known in the art. For example, such leads may enter at an end 15 of header block 14 and be physically and electrically connected to conductive receptacles or other conductive features located within header block 14. IMD 10 is adapted to be implanted subcutaneously in the body of a patient such that it becomes encased within body tissue and fluids, which may include epidermal layers, subcutaneous fat layers, and/or muscle layers.

Hermetically sealed housing 12 is generally circular, elliptical, prismatic, or rectilinear, with substantially planar major sides (only one major side 16 is shown in FIG. 1) joined by perimeter sidewalls. The perimeter sidewalls include a substantially straight first sidewall 18, a substantially straight second sidewall 20 opposing first sidewall 18, a substantially straight upper sidewall 22, and a curvilinear lower sidewall 24 opposing upper sidewall 22. Housing 12 is typically formed from pieces of a thin-walled biocompatible metal such as titanium. Two half sections of housing 12 may be laser seam welded together using conventional techniques to form a seam extending around the perimeter sidewalls.

Housing 12 and header block 14 are often manufactured as two separate assemblies that are subsequently physically and electrically coupled together. Housing 12 may contain a number of functional elements, components, and features, including (without limitation): a battery; a high voltage output capacitor; integrated circuit (“IC”) devices; a processor; memory elements; a therapy module or circuitry; an RF module or circuitry; and an antenna matching circuit. These components may be assembled in spacers and disposed within the interior cavity of housing 12 prior to seam welding of the housing halves. During the manufacturing process, electrical connections are established between components located within housing 12 and elements located within header block 14. For example, housing 12 and header block 14 may be suitably configured with IC connector pads, terminals, feedthrough pins, and other features for establishing electrical connections between the internal therapy module and the therapy lead connectors within header block 14 and for establishing connections between the internal RF module and a telemetry antenna located within header block 14. Structures and techniques for establishing such electrical (and physical) connections are known to those skilled in the art and, therefore, will not be described in detail herein.

Header block 14 is preferably formed from a suitable dielectric material, such as a biocompatible synthetic polymer, tecothane, ceramic, or biocompatible glass. The dielectric material of header block 14 passes RF energy that is either radiated or received by a telemetry antenna (not shown in FIG. 1) encapsulated within header block 14. The encapsulation of the antenna within header block 14 insulates the antenna from the tissue and fluids after implantation. In practical embodiments, header block 14 is formed from a material having a relative dielectric constant of approximately 3.0 to 5.0. The specific material for header block 14 may be chosen in response to the intended application of IMD 10, the electrical characteristics of the environment surrounding the implant location, the desired operating frequency range, the desired RF antenna range, and other practical considerations.

In accordance with one example embodiment, header block 14 is approximately one inch wide (measured along upper sidewall 22), approximately one-half inch high, and approximately one-half inch thick. It should be appreciated that the shape, size, topology, and placement of header block 14 relative to housing 12 may vary from one application to another, and that the particular configuration shown in FIG. 1 represents only one practical example. In this regard, header block 14 may, but need not, have a “tail” 26 that extends partially down sidewall 20. Alternate embodiments may include a longer or shorter tail 26, depending upon the desired locations of electrical connections and interface points, or depending upon the layout and routing of conductive elements contained within header block 14 and tail 26. In addition, header block 14 need not be located on upper sidewall 22 (or any sidewall) and may instead be located on one of the planar major sides of housing 12. Furthermore, more than one header block 12 may be utilized in a practical implementation.

FIG. 2 is a schematic representation of an IMD 100 and several functional elements associated therewith. IMD 100 generally includes a housing 102, a header block 104 coupled to housing 102, a therapy module 106 contained within housing 102, an RF module 108 contained within housing 102, an RF impedance matching circuit 110, which may also be contained within housing 102, and a telemetry antenna 112 that is suitably configured to facilitate far field data communication with an EMD. Housing 102 and header block 104 may be configured as described above in connection with FIG. 1. In practice, IMD 100 will also include a number of conventional components and features necessary to support the functionality of IMD 100. Such conventional elements will not be described herein.

Therapy module 106 may include any number of components, including, without limitation: electrical devices, ICs, microprocessors, controllers, memories, power supplies, and the like. Briefly, therapy module 106 is configured to provide the desired functionality associated with the IMD 100, e.g., defibrillation pulses, pacing stimulation, patient monitoring, or the like. In this regard, therapy module 106 may be coupled to one or more therapy lead connectors 114, which may be located within header block 104. In turn, therapy lead connectors 114 are electrically coupled to therapy leads (not shown) that extend from header block 104 for routing and placement within the patient.

RF module 108 may include any number of components, including, without limitation: electrical devices, ICs, amplifiers, signal generators, a receiver and a transmitter (or a transceiver), modulators, microprocessors, controllers, memories, power supplies, and the like. Although matching circuit 110 is illustrated as a separate component coupled to RF module 108, it may instead be incorporated into RF module 108 in a practical embodiment. Briefly, RF module 108 supports RF telemetry communication for IMD 100, including, without limitation: generating RF transmit energy; providing RF transmit signals to antenna 112; processing RF telemetry signals received by antenna 112, and the like. In practice, RF module 108 may be designed to leverage the conductive material used for housing 102 as an RF ground, and RF module 108 may be designed in accordance with the intended application of IMD 100, the electrical characteristics of the environment surrounding the implant location, the desired operating frequency range, the desired RF antenna range, and other practical considerations.

Matching circuit 110 may include any number of components, including, without limitation: electrical components such as capacitors, resistors, or inductors; filters; baluns; tuning elements; varactors; limiter diodes; or the like. Matching circuit 110 is suitably configured to provide impedance matching between antenna 112 and RF module 108, thus improving the efficiency of antenna 112. Matching circuit 110 may leverage known techniques to alter the electrical characteristics of antenna 112 to suit the needs of the particular application. For example, matching circuit 110 may be suitably configured to enhance the far field radiation characteristics of antenna 112 while allowing antenna 112 to be physically compact and conformal for practical deployment in an IMD 100 having relatively strict physical size limitations.

RF module 108 and/or matching circuit 110 may also be configured to support the particular design and intended operation of antenna 112. For example, antenna 112 may have characteristics resembling a monopole antenna, characteristics resembling a dipole antenna, characteristics resembling a coplanar waveguide antenna, characteristics resembling a stripline antenna, characteristics resembling a microstrip antenna, and/or characteristics resembling a transmission line antenna. Antenna 112 may also have any number of radiating elements, which may be driven by any number of distinct RF signal sources. In this regard, antenna 112 may have a plurality of radiating elements configured to provide spatial or polarization diversity. In view of the different practical options for antenna 112, RF module 108 and/or matching circuit 110 can be customized in an appropriate manner.

Antenna 112 is coupled to matching circuit 110 and/or to RF module 108 to facilitate RF telemetry between IMD 100 and an EMD (not shown). Generally, antenna 112 is suitably configured for UHF or VHF operation. In the example embodiment of the invention, antenna 112 is located within header block 104. In practice, antenna 112 may be encapsulated by the dielectric material used to form header block 104. Antenna 112 (or at least a radiating element of antenna 112) is coupled to matching circuit 110 and/or to RF module 108 via an RF feedthrough 116, which bridges housing 102. Briefly, a practical RF feedthrough 116 includes a ferrule supporting a non-conductive glass or ceramic annular insulator. The insulator supports and electrically isolates a feedthrough pin from the ferrule. During assembly of housing 102, the ferrule is welded to a suitably sized hole or opening formed in housing 102. Matching circuit 110 and/or RF module 108 is then electrically connected to the inner end of the feedthrough pin. The connection to the inner end of the feedthrough pin can be made by welding the inner end to a substrate pad, or by clipping the inner end to a cable or flex wire connector that extends to a substrate pad or connector. The outer end of the feedthrough pin serves as a connection point for antenna 112.

In FIG. 2, RF feedthrough 116 is located on the upper perimeter sidewall of housing 102 such that it defines a feed point for antenna 112, leading from housing 102 into header block 104. Alternatively, RF feedthrough 116 may be located on the lower perimeter sidewall of housing 102, on either of the major perimeter sidewalls of housing 102, or on either of the major sides of housing 102. Consequently, any of the antenna arrangements described herein may be modified to accommodate different RF feedthrough locations. For example, a given antenna may utilize an input section that leads from the RF feedthrough location to the main section of the header block. Furthermore, depending upon the specific configuration and topology of antenna 112, a single RF feedthrough may provide insulated routing for any number of separate radiating elements, and/or IMD 100 may include any number of separate RF feedthroughs for a like number of separate antenna elements.

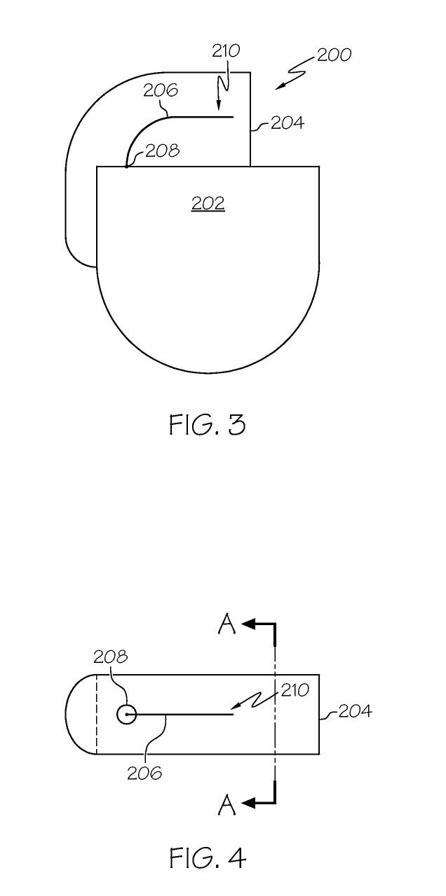

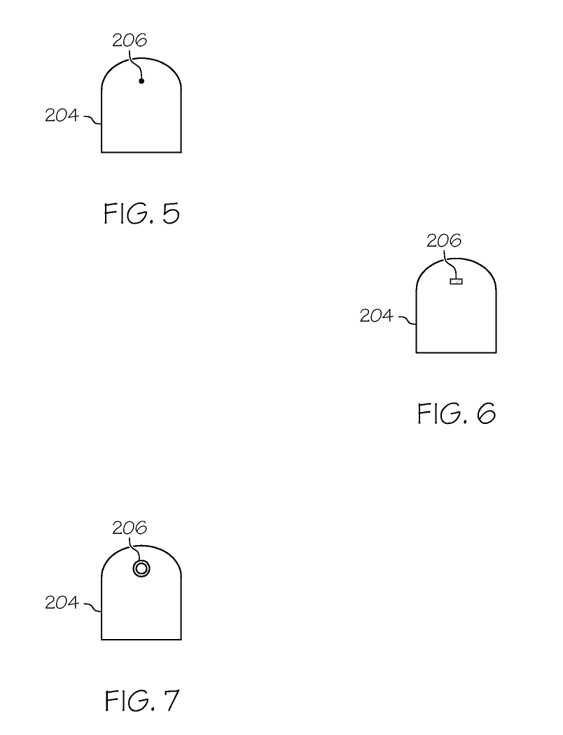

FIG. 3 is a side view of an IMD 200 configured in accordance with an example embodiment of the invention, FIG. 4 is a top view of the header block portion of IMD 200, and FIG. 5 is a cross sectional view of the header block portion as viewed along line A-A in FIG. 4. Certain features and aspects of IMD 200 are similar to those described above in connection with IMD 10 and IMD 100, and shared features and aspects will not be redundantly described in the context of IMD 200. IMD 200 generally includes a housing 202, a header block 204 coupled to housing 202, and an antenna 206. In this particular embodiment, antenna 206 is completely contained within header block 204. In practice, antenna 206 is encapsulated within the dielectric material that forms header block 204. Antenna 206 makes electrical contact with an RF feedthrough 208 when header block 204 is coupled to housing 202. In accordance with known techniques, the conductive element of antenna 206 may be attached to the feedthrough pin via welding, and a biocompatible medical adhesive or epoxy may be used to cover and electrically insulate any exposed portions of the feedthrough pin or the conductive element of the antenna.

Antenna 206 is preferably dimensioned and otherwise configured to fit within the space limitations of header block 204. In addition, antenna 206 is dimensioned to provide far field radiation of RF transmit energy provided by the RF module contained within housing 202. In accordance with one practical application, antenna 206 is suitably dimensioned and tuned for reception and transmission of RF signals having a carrier frequency within the range of 402 MHz to 405 MHz. Antenna 206 is preferably dimensioned and tuned to account for the intended operating environment (IMD 200 is surrounded by conductive body tissue when deployed) and to account for the desired far field operating range. In this regard, antenna 206 is preferably designed to meet system requirements for a two-meter minimum telemetry range and to provide adequate gain, gain pattern, bandwidth, and tunability using one or more reactive elements for different possible environments before and after implanting of IMD 200.

As shown in FIG. 3, antenna 206 is shaped such that its profile forms a simple curve (when viewed from the perspective of FIG. 3). The curved shape enables antenna 206 to assume a compact form within header block 204 while maintaining the desired electrical length necessary for good far field telemetry performance. Antenna 206 is “open ended” such that it has a floating endpoint 210 in header block 204. This configuration allows antenna 206 to have unbalanced operating characteristics that resemble a simple monopole antenna. Antenna 206 may be curved such that it defines a plane that is generally parallel to a major side or sides of housing 202 and/or generally parallel to a major side or sides of header block 204 (see FIG. 4). In the example embodiment, antenna 206 is evenly spaced between the two major sides of header block 204 and evenly spaced between the two major sides of housing 202, thus eliminating a potential source of asymmetry. In one practical embodiment of the invention, antenna 206 extends across header block 204 and is positioned as far away from housing 202 as possible while still being insulated from the body tissue.

To implement effective telemetry from a given IMD over the desired distances, the driving power should be efficiently converted to maximize the far field component generated by antenna 206. One factor affecting the far field component is the length of antenna 206 with respect to the wavelength of the radiating RF carrier signal. While many types of antennas function according to a variety of parameters, it is generally desirable to provide an antenna having a minimum length equivalent to one-quarter or one-half the wavelength of the RF carrier signal. Longer lengths typically provide better performance and the overall length is preferably a multiple of the half wavelength of the carrier signal. Other factors include the dielectric values imposed by the surrounding medium, e.g., housing 202, header block 204, and the surrounding patient environment.

Antenna 206 may include a radiating element formed from a conductive wire, such as a titanium wire, a niobium wire, or the like. As shown in the cross sectional view of FIG. 5, antenna 206 may be formed from a solid wire having a round cross section. In practical embodiments, antenna 206 may be formed from a round wire having a diameter of approximately 0.020 inches. Alternatively, as depicted in FIG. 6, antenna 206 may be formed from a flat wire, a flat ribbon element, or a stamped conductor having a generally rectangular cross section (or, for that matter, any practical cross sectional shape). FIG. 7 depicts yet another embodiment where antenna 206 is formed from a hollow wire having a round ring shaped cross section. FIG. 3 equivalently depicts any embodiment that employs an antenna having a relatively thin profile or height, and FIG. 4 equivalently depicts any embodiment that employs a relatively thin wire for antenna 206.

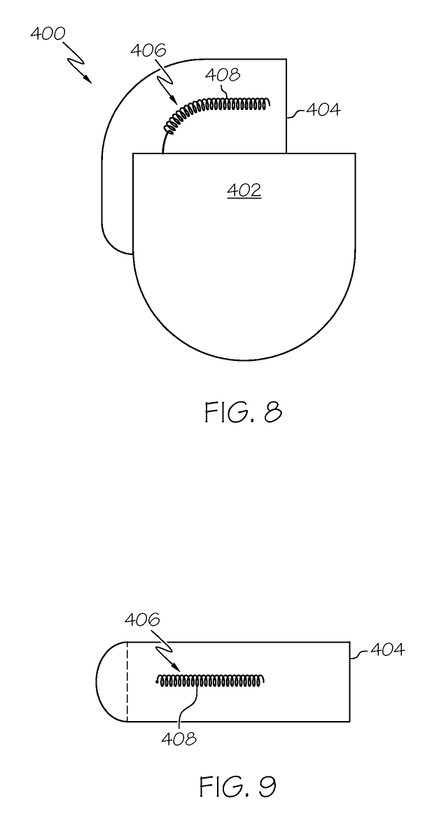

FIG. 8 is a side view of an IMD 400 configured in accordance with another alternate embodiment of the invention, and FIG. 9 is a top view of the header block portion of IMD 400. Certain features and aspects of IMD 400 are similar to those described above in connection with IMD 10, IMD 100, and IMD 200, and shared features and aspects will not be redundantly described in the context of IMD 400. IMD 400 generally includes a housing 402, a header block 404 coupled to housing 402, and an antenna 406 located within header block 404. The feed point of antenna 406 may, but need not be, on the upper perimeter sidewall of housing 402. Antenna 406 includes a radiating element 408 comprising a helical coil. In practice, any portion of radiating element 408 may be formed from a helical coil section, and more than one helical coil section may be utilized in radiating element 408. The helical coil section allows the overall length of antenna 406 to be relatively short while maintaining the necessary electrical length.

In the example embodiment, antenna 406 has a simple curved profile, as shown in FIG. 8. In this regard, the relative positioning of antenna 406 is similar to that of antenna 206 described above. Although antenna 406 is depicted as an open ended element, an alternate embodiment may have a grounded endpoint rather than a floating endpoint, thus forming a balanced loop antenna structure.

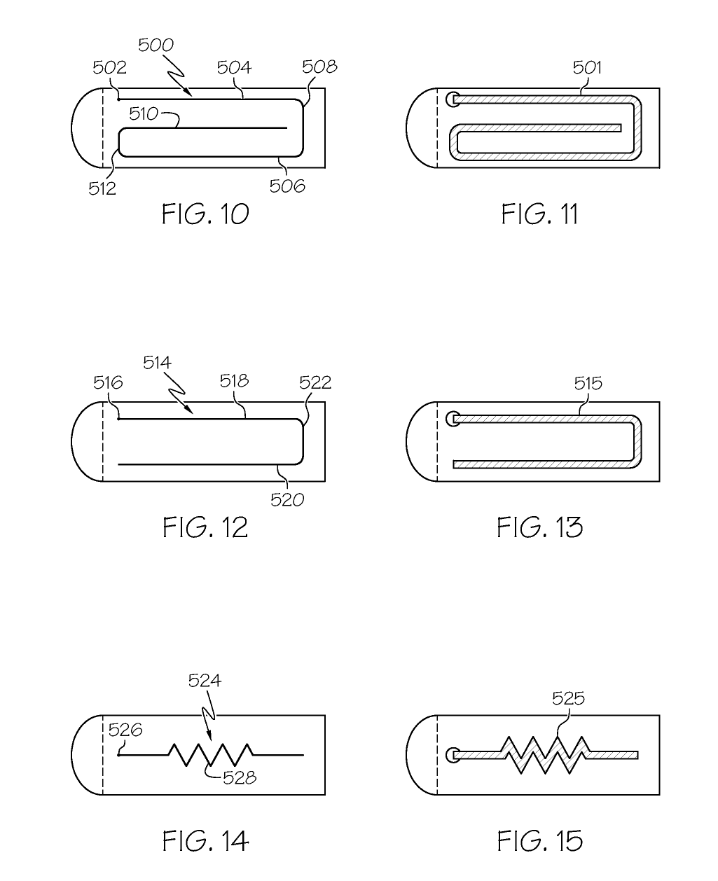

FIGS. 10-15 are top views of the header block portion of IMDs configured in accordance with alternate embodiments of the invention. Although not required, each of these antennas may have a side profile that defines a simple curve (see antenna 206 shown in FIG. 3). Alternatively, the endpoints of any of these antennas may be grounded (forming a loop antenna) rather than floating as shown. Furthermore, each of these antennas may, but need not, have a feed point on the upper perimeter sidewall of the IMD housing.

FIG. 10 depicts an antenna 500 having a thin wire radiating element that resembles a paper clip from the top view of the IMD (FIG. 11 depicts a similar antenna 501 that is formed from a flat wire or ribbon radiating element rather than a thin wire radiating element). The radiating element of antenna 500 comprises a first end 502 defining a feed point for antenna 500, and a first radiating section 504 coupled to or in communication with first end 502. The radiating element of antenna 500 also includes a second radiating section 506 and a first bend section 508 coupling first radiating section 504 to second radiating section 506. As shown in FIG. 10, first bend section 508 is U-shaped in the example embodiment. The radiating element of antenna 500 further includes a third radiating section 510 and a second bend section 512 coupling second radiating section 506 to third radiating section 510. Second bend section 512 is also U-shaped in the example embodiment. It should be appreciated that the various sections of the radiating element may be realized from a single continuous piece of material. The topology of antenna 500 is such that the direction of surface current in first radiating section 504 opposes the direction of surface current in second radiating section 506 and such that the direction of surface current in second radiating section 506 opposes the direction of surface current in third radiating section 510. In operation, this topology reduces the potentially negative effect of surface current cancellation, thus improving the efficiency of antenna 500.

FIG. 12 depicts an antenna 514 having a thin wire radiating element that resembles a “U” from the top view of the IMD (FIG. 13 depicts a similar antenna 515 that is formed from a flat wire or ribbon radiating element rather than a thin wire radiating element). The radiating element of antenna 514 comprises a first end 516 defining a feed point for antenna 514, and a first radiating section 518 coupled to or in communication with first end 516. The radiating element of antenna 514 also includes a second radiating section 520 and a bend section 522 coupling first radiating section 518 to second radiating section 520. As shown in FIG. 12, bend section 522 is U-shaped in the example embodiment. It should be appreciated that the various sections of the radiating element may be realized from a single continuous piece of material. The topology of antenna 514 is such that the direction of surface current in first radiating section 518 opposes the direction of surface current in second radiating section 520. In operation, this topology reduces the potentially negative effect of surface current cancellation, thus improving the efficiency of antenna 514.

FIG. 14 depicts an antenna 524 having a thin wire radiating element that resembles a saw tooth wave from the top view of the IMD (FIG. 15 depicts a similar antenna 525 that is formed from a flat wire or ribbon radiating element rather than a thin wire radiating element). The radiating element of antenna 524 comprises a first end 526 defining a feed point for antenna 524 and a saw tooth radiating section 528 coupled to or in communication with first end 502. It should be appreciated that the various sections of the radiating element may be realized from a single continuous piece of material. This configuration results in less surface current cancellation in antenna 524 and, therefore, enhanced RF performance.

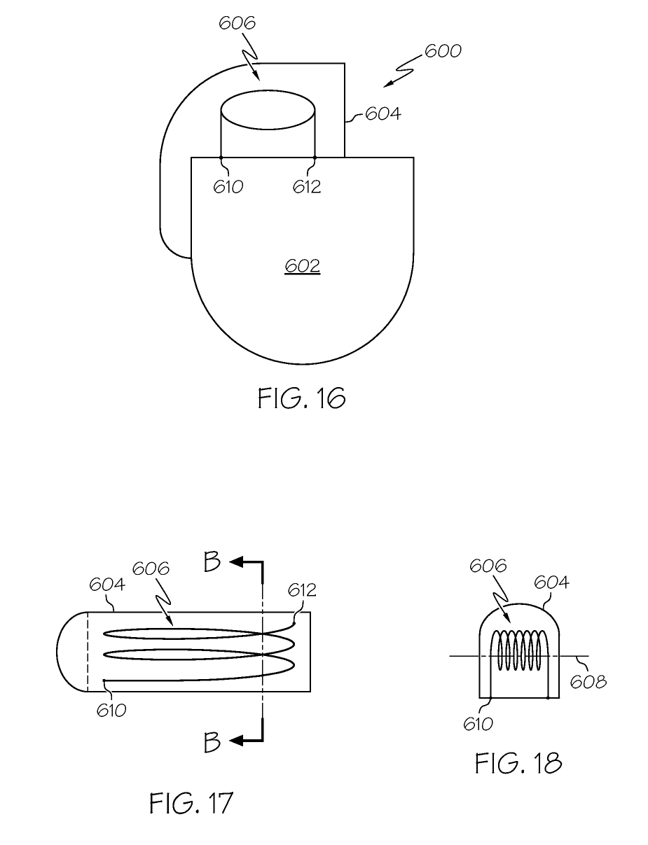

FIG. 16 is a side view of an IMD 600 configured in accordance with an alternate embodiment of the invention, FIG. 17 is a top view of the header block portion of IMD 600, and FIG. 18 is a cross sectional view of the header block portion of IMD 600, as viewed along line B-B in FIG. 17. Certain features and aspects of IMD 600 are similar to those described above in connection with IMD 10 and IMD 100, and shared features and aspects will not be redundantly described in the context of IMD 600. IMD 600 generally includes a housing 602, a header block 604 coupled to housing 602, and an antenna 606. In this example, antenna 606 is a loop antenna having a grounded endpoint, where the loops generally wrap around an imaginary axis 608 (see FIG. 18) that runs approximately perpendicular to the major sides of housing 602. Antenna 606 may be formed from a thin wire radiating element (as shown) or from a flat ribbon, flat wire, or stamped metal radiating element. In the example embodiment, antenna 606 forms multiple loops within header block 604.

Antenna 606 has a first end 610 and a second end 612. First end 610 may define a feed point for antenna 606 and second end 612 may define a ground point for antenna 606. In this regard, the ground point may be coupled to housing 602 or to a suitable RF ground point on the RF module contained within housing 602. Of course, an alternate embodiment may have first end 610 serving as RF ground and second end 612 serving as the RF feed point. Furthermore, the relative positioning of the RF ground point and the RF feed point may vary depending upon the practical deployment. In practice, if antenna 606 is centered within header block 604, as depicted in FIG. 17 and FIG. 18, then IMD 600 will have a more symmetrical radiation pattern. Otherwise, if antenna 606 is offset within header block 604, then the radiation pattern would be biased toward one major side of housing 602.

In operation, antenna 606 functions as a magnetic loop antenna having lower wave impedance, higher current, and lower voltage in the near field, but better far field radiation compared to lower frequency coils, such as transformers. In yet another practical embodiment, two antennas 606, having opposing RF feed and ground points, can be utilized to form a balanced antenna arrangement for IMD 600.

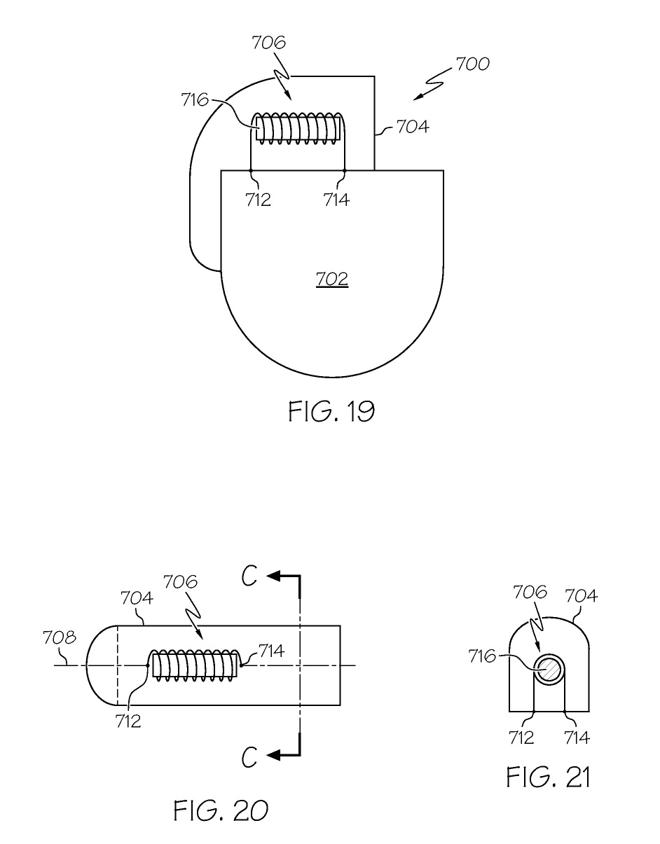

FIG. 19 is a side view of an IMD 700 configured in accordance with another alternate embodiment of the invention, FIG. 20 is a top view of the header block portion of IMD 700, and FIG. 21 is a cross sectional view of the header block portion of IMD 700, as viewed along line C-C in FIG. 20. Certain features and aspects of IMD 700 are similar to those described above in connection with IMD 10 and IMD 100, and shared features and aspects will not be redundantly described in the context of IMD 700. IMD 700 generally includes a housing 702, a header block 704 coupled to housing 702, and an antenna 706. In this example, antenna 706 is a loop antenna having a grounded endpoint, where the loops generally wrap around an imaginary axis 708 (see FIG. 20) that runs approximately parallel to the major sides of housing 702. Antenna 706 may include a thin wire radiating element (as shown) or, alternatively, a flat ribbon, flat wire, or stamped metal radiating element. In the example embodiment, antenna 706 forms multiple loops within header block 704. In practice, antenna 706 forms a tuned circuit coil, and IMD 700 includes an LRC tuning circuit configured to tune antenna 706 such that it resonates at the desired frequency. The LRC circuit, which is coupled to antenna 706, is preferably contained within housing 702.

Antenna 706 has a first end 712 and a second end 714. First end 712 may define a feed point for antenna 706 and second end 714 may define a ground point for antenna 706. In this regard, the ground point may be coupled to housing 702 or to a suitable RF ground point on the RF module contained within housing 702. Of course, an alternate embodiment may have first end 712 serving as RF ground and second end 714 serving as the RF feed point. Furthermore, the relative positioning of the RF ground point and the RF feed point may vary depending upon the practical deployment.

In accordance with the example embodiment, antenna 706 is wrapped around a core 716 formed from a suitable biocompatible material having a high permeability. For example, ferrite or another ferromagnetic material may be suitable for use as core 716. Core 716 may be realized as a metal rod encapsulated by a dielectric material such as a ceramic composition. Core 716 may be desirable to enhance the efficiency and performance of antenna 706 relative to a version having no core 716.

While at least one example embodiment has been presented in the foregoing detailed description, it should be appreciated that a vast number of variations exist. It should also be appreciated that the example embodiment or embodiments described herein are not intended to limit the scope, applicability, or configuration of the invention in any way. Rather, the foregoing detailed description will provide those skilled in the art with a convenient road map for implementing the described embodiment or embodiments. It should be understood that various changes can be made in the function and arrangement of elements without departing from the scope of the invention as set forth in the appended claims and the legal equivalents thereof.

What is claimed is:

1. An implantable medical device (“IMD”) comprising:
a housing;
a header block coupled to said housing; and
a curved antenna located within said header block, said antenna having a feed point from said housing leading into said header block, and said antenna having a floating endpoint in said header block.

2. An IMD according to claim 1, said antenna being formed from a round wire.

3. An IMD according to claim 1, said antenna being formed from a flat ribbon.

4. An IMD according to claim 1, said antenna being formed from a hollow wire.

5. An IMD according to claim 1, said antenna defining a plane that is generally parallel to a major side of said header block.

6. An IMD according to claim 1, said antenna defining a plane that is generally parallel to a major side of said housing.

7. An IMD according to claim 1, said feed point being located on a perimeter sidewall of said housing.

8. An IMD according to claim 1, said feed point being located on a major side of said housing.

9. An IMD according to claim 1, said antenna being evenly spaced between major sides of said header block.

10. An IMD according to claim 1, said header block being formed from a dielectric material, and said antenna being encapsulated by said dielectric material.

11. An IMD according to claim 1, further comprising a radio frequency (“RF”) module contained in said housing and coupled to said antenna, said antenna being dimensioned to provide far field radiation of RF transmit energy provided by said RF module when the IMD is implanted in a human body.

12. An IMD according to claim 11, further comprising a radio frequency (“RF”) impedance matching circuit coupled to said antenna, said RF impedance matching circuit being configured to match said antenna to said RF module.

13. An IMD according to claim 12, said RF impedance matching circuit being contained in said housing.

14. A far field telemetry antenna for an implantable medical device (“IMD”) having a dielectric header block, said antenna comprising a radiating element having:
a first end defining a feed point for said antenna; and
a second end defining a floating endpoint for said antenna;
said radiating element being configured with a simple-curved profile and being dimensioned to fit within said dielectric header block.

15. A far field telemetry antenna according to claim 14, said radiating element being formed from a wire.

16. A far field telemetry antenna according to claim 15, said radiating element defining a plane that is generally parallel to a major side of said dielectric header block.

17. A far field telemetry antenna according to claim 14, said radiating element being formed from a ribbon element.

18. A far field telemetry antenna according to claim 14, said radiating element comprising a helical coil.

19. A far field telemetry antenna according to claim 14, said radiating element comprising a first radiating section coupled to said first end, a second radiating section parallel to said first radiating section, and a bend section coupling said first radiating section to said second radiating section.

20. A far field telemetry antenna according to claim 19, said bend section being U-shaped.

21. A far field telemetry antenna according to claim 20, said radiating element further comprising a third radiating section parallel to said first radiating section and parallel to said second radiating section, and a second bend section coupling said second radiating section to said third radiating section.

22. A far field telemetry antenna according to claim 21, said second bend section being U-shaped.
